(19) United States
(12) Patent Application Publication
(10) Pub. No.: US 2002/0153507 A1
(43) Pub. Date: Oct. 24, 2002

(54) Use of phosphate esters to extend the pot-life of isocyanates, isocyanate pre-polymers and blends

(76) Inventors: Thomas G. Savino, Northville, MI (US); Nikolay Lebedinski, West Bloomfield, MI (US)

Correspondence Address: BASF CORPORATION, LEGAL DEPARTMENT, 1609 BIDDLE AVENUE, WYANDOTTE, MI, 48192, US

(21) Appl. No.: 09/789,777

(22) Filed: Feb. 20, 2001

(51) Int. Cl. C08K 5/521; C07C 265/00; C08G 18/70; C09K 15/32

(52) U.S. Cl. 252/182.2; 252/182.21; 252/182.22; 252/182.29; 560/333

(57) ABSTRACT

An isocyanate prepolymer composition having an extended potlife, without the addition of an excess amount of benzoyl chloride is disclosed. The isocyanate prepolymer composition includes an isocyanate prepolymer formed from the reaction of a polyhydroxyl compound with an excess of a polyisocyanate. The composition further includes included a plasticizer, and a phosphate ester. The phosphate ester has at least one acidic hydrogen and a pentavalent phosphorous atom. Preferably, the composition also includes very low levels of benzoyl chloride and an oil. Further disclosed is a polyisocyanate composition having an extended potlife comprising a polyisocyanate, a plasticizer and a phosphate ester having at least one acidic hydrogen and a pentavalent phosphorous atom.

### FIELD OF THE INVENTION

The present invention pertains to isocyanates, isocyanate blends, isocyanate pre-polymers, isocyanate prepolymer blends and the use of phosphate esters to extend the time period during which the isocyanates, isocyanate pre-polymers, or their blends may be used in a processing operation. With more particularity, the invention pertains to isocyanates and isocyanate pre-polymers and the use of esters of phosphoric acid to restrict the reaction of the isocyanate with ambient water in the air and thereby extend the time period in which the isocyanate or isocyanate pre-polymer may be used in a processing operation.

### BACKGROUND OF THE INVENTION

Polyurethane prepolymers have wide spread use in the manufacturing industry. For example, polyurethane prepolymers may be used as adhesives and are commonly utilized in the production of thermoformable polyurethane foam products. Typically, layers of slab stock foam are coated with an adhesive and are then pressed together at an elevated temperature and pressure to form a thermoformed article. A common polyurethane adhesive consists of a blend of isocyanates or isocyanate prepolymers that have residual NCO groups. A problem with known polyurethane isocyanates and prepolymer compositions is the limited manufacturing time or “potlife” due to the reaction of the NCO groups with water from humidity in the ambient air.

A known “potlife” extender that is utilized in the industry is benzoyl chloride. Benzoyl chloride has several detrimental properties including: toxicity to both humans and animals; it is an irritant; and it has an offensive odor. These properties of benzoyl chloride usually preclude its use in amounts greater than .02 weight percent based on the weight of the isocyanate or prepolymer and thereby limits the amount of time the “potlife” may be extended. It would be desirable to extend the “potlife” of an isocyanate or isocyanate prepolymer without the negative properties associated with the use of benzoyl chloride.

### SUMMARY OF THE INVENTION

The present invention provides an isocyanate or isocyanate prepolymer composition including a phosphate ester that has an extended potlife, without the addition of an excess amount of benzoyl chloride. The isocyanate prepolymer composition is formed from the reaction of a polyhydroxyl compound and an excess of an isocyanate compound. There may also be included a plasticizer, benzoyl chloride and an oil. A phosphate ester is also included as a component of the prepolymer composition. A phosphate ester as the term is used in the present specification and claims has the following characteristics: at least one acidic hydrogen; a pentavalent phosphorous atom; and at least one ester group, which may comprise an alkyl, aryl, aliphatic, or cycloaliphatic substituent.

### DETAILED DESCRIPTION OF THE PREFERRED EMBODIMENT

An isocyanate prepolymer composition of the present invention is formed from the reaction of a polyhydroxyl compound, an isocyanate compound, a plasticizer, and a phosphate ester. It is to be understood that the present invention can also be utilized to extend the potlife of blends of isocyanate prepolymers.

The polyhydroxyl compound may be either a polyester or polyether polyol.

The term “polyester polyol” as used in this specification and claims includes any minor amounts of unreacted polyol remaining after the preparation of the polyester polyol and/or unesterified polyol (e.g., glycol) added after the preparation of the polyester polyol. The polyester polyol can include up to about 40 weight percent free glycol.

Suitable polyester polyols can be produced, for example, from organic dicarboxylic acids with 2 to 12 carbons, preferably aliphatic dicarboxylic acids with 4 to 6 carbons, and multivalent alcohols, preferably diols, with 2 to 12 carbons, preferably 2 to 6 carbons. Examples of dicarboxylic acids include succinic acid, glutaric acid, adipic acid, suberic acid, azelaic acid, sebacic acid, decanedicarboxylic acid, maleic acid, fumaric acid, phthalic acid, isophthalic acid and terephthalic acid. The dicarboxylic acids can be used individually or in mixtures. Instead of the free dicarboxylic acids, the corresponding dicarboxylic acid derivatives may also be used such as dicarboxylic acid mono- or di-esters of alcohols with 1 to 4 carbons, or dicarboxylic acid anhydrides. Dicarboxylic acid mixtures of succinic acid, glutaric acid and adipic acid in quantity ratios of 20-35:-35-50:20-32 parts by weight are preferred, especially adipic acid. Examples of divalent and multivalent alcohols, especially diols, include ethanediol, diethylene glycol, 1,2- and 1,3-propanediol, dipropylene glycol, 1,4-butanediol, 1,5-pentanediol, 1,6-hexanediol, 1,10-decanediol, glycerine and trimethylolpropanes, tripropylene glycol, tetraethylene glycol, tetrapropylene glycol, tetramethylene glycol, 1,4-cyclohexane-dimethanol, ethanediol, diethylene glycol, 1,4-butanediol, 1,5-pentanediol, 1,6-hexanediol or mixtures of at least two of these diols are preferred, especially mixtures of 1,4-butanediol, 1,5-pentanediol and 1,6-hexanediol. Furthermore, polyester polyols of lactones, e.g., ε-caprolactone or hydroxycarboxylic acids, e.g., ω-hydroxycaproic acid, may also be used.

The polyester polyols can be produced by polycondensation of organic polycarboxylic acids, e.g., aromatic or preferably aliphatic polycarboxylic acids and/or derivatives thereof and multivalent alcohols in the absence of catalysts or preferably in an atmosphere of inert gases, e.g., nitrogen, carbon dioxide, helium, argon, etc., in the melt at temperatures of 150° to 250° C., preferably 180° to 220° C., optionally under reduced pressure, up to the desired acid value which is preferably less than 10, especially less than 2. In a preferred embodiment, the esterification mixture is subjected to polycondensation at the temperatures mentioned above up to an acid value of 80 to 30, preferably 40 to 30, under normal pressure, and then under a pressure of less than 500 mbar, preferably 50 to 150 mbar. The reaction can be carried out as a batch process or continuously. When present, excess glycol can be distilled from the reaction mixture during and/or after the reaction, such as in the preparation of low free glycol-containing polyester polyols usable in the present invention.

Examples of suitable esterification catalysts include iron, cadmium, cobalt, lead, zinc, antimony, magnesium, titanium and tin catalysts in the form of metals, metal oxides or metal salts. However, the polycondensation may also be preformed in liquid phase in the presence of diluents and/or chlorobenzene for aziotropic distillation of the water of condensation.

To produce the polyester polyols, the organic polycarboxylic acids and/or derivatives thereof and multi-valent alcohols are preferably polycondensed in a mole ratio of 1:1-1.8, more preferably 1:1.05-1.2.

After transesterification or esterification, the reaction product can be reacted with an alkylene oxide to form a polyester polyol mixture. This reaction desirably is catalyzed. The temperature of this process should be from about 80° to 170° C., and the pressure should generally range from about 1 to 40 atmospheres.

While the aromatic polyester polyols can be prepared from substantially pure reactant materials, more complex ingredients can be used, such as the side stream, waste or scrap residues from the manufacture of phthalic acid, terephthalic acid, dimethyl terephthalate, polyethylene terephthalate, and the like. Compositions containing phthalic acid residues for use in the invention are (a) ester-containing byproducts from the manufacture of dimethyl terephthalate, (b) scrap polyalkylene terephthalates, (c) phthalic anhydride, (d) residues from the manufacture of phthalic acid or phthalic anhydride, (e) terephthalic acid, (f) residues from the manufacture of terephthalic acid, (g) isophthalic acid, (h) trimellitic anhydride, and (i) combinations thereof. These compositions may be converted by reaction with the polyols of the invention to polyester polyols through conventional transesterification or esterification procedures.

Other materials containing phthalic acid residues are polyalkylene terephthalates, especially polyethylene terephthalate (PET), residues or scraps. Still other residues are DMT process residues, which are waste or scrap residues from the manufacture of dimethyl terephthalate (DMT). The term “DMT process residue” refers to the purged residue which is obtained during the manufacture of DMT in which p-xylene is converted through oxidation and esterification with methanol to the desired product in a reaction mixture along with a complex mixture of byproducts. The desired DMT and the volatile methyl p-toluate byproduct are removed from the reaction mixture by distillation leaving a residue. The DMT and methyl p-toluate are separated, the DMT is recovered and methyl p-toluate is recycled for oxidation. The residue which remains can be directly purged from the process or a portion of the residue can be recycled for oxidation and the remainder diverted from the process or, if desired, the residue can be processed further as, for example, by distillation, heat treatment and/or methanolysis to recover useful constituents which might otherwise be lost, prior to purging the residue from the system. The residue which is finally purged from the process, either with or without additional processing, is herein called DMT process residue.

Polyoxyalkylene polyether polyols, which can be obtained by known methods, are preferred for use as the polyhydroxyl compound of the isocyanate prepolymer composition. For example, polyether polyols can be produced by anionic polymerization with alkali hydroxides such as sodium hydroxide or potassium hydroxide or alkali alcoholates, such as sodium methylate, sodium ethylate, or potassium ethylate or potassium isopropylate as catalysts and with the addition of at least one initiator molecule containing 2 to 8, preferably 3 to 8, reactive hydrogens or by cationic polymerization with Lewis acids such as antimony pentachloride, boron trifluoride etherate, etc., or bleaching earth as catalysts from one or more alkylene oxides with 2 to 4 carbons in the alkylene radical. Any suitable alkylene oxide may be used such as 1,3-propylene oxide, 1,2-and 2,3-butylene oxide, amylene oxides, styrene oxide, and preferably ethylene oxide and 1,2-propylene oxide and mixtures of these oxides. The polyalkylene polyether polyols may be prepared from other starting materials such as tetrahydrofuran and alkylene oxide-tetrahydrofuran mixtures; epihalohydrins such as epichlorohydrin; as well as aralkylene oxides such as styrene oxide. The polyalkylene polyether polyols may have either primary or secondary hydroxyl groups.

Included among the polyether polyols are polyoxyethylene glycol, polyoxypropylene glycol, polyoxybutylene glycol, polytetramethylene glycol, block copolymers, for example, combinations of polyoxypropylene and polyoxyethylene glycols, poly-1,2-oxybutylene and polyoxyethylene glycols, poly-1,4-tetramethylene and polyoxyethylene glycols, and copolymer glycols prepared from blends or sequential addition of two or more alkylene oxides. The polyalkylene polyether polyols may be prepared by any known process such as, for example, the process disclosed by Wurtz in 1859 and Encyclopedia of Chemical Technology, Vol. 7, pp. 257-262, published by Interscience Publishers, Inc. (1951) or in U.S. Pat. No. 1,922,459.

Polyethers which are preferred include the alkylene oxide addition products of polyhydric alcohols such as ethylene glycol, propylene glycol, dipropylene glycol, trimethylene glycol, 1,2-butanediol, 1,5-pentanediol, 1,6-hexanediol, 1,7-heptanediol, hydroquinone, resourcinol glycerol, glycerine, 1,1,1-trimethylol-propane, 1,1,1-trimethylolethane, pentaerythritol, 1,2,6-hexanetriol, a-methyl glucoside, sucrose, and sorbitol. Also included within the term “polyhydric alcohol” are compounds derived from phenol such as 2,2-bis(4-hydroxyphenyl)-propane, commonly known as Bisphenol A.

A particularly preferred polyether polyol of the present invention includes Pluracol® 380, a TMP (trimethylolpropane) initiated propylene and ethylene oxide polyether polyol commercially available from BASF Corporation.

The isocyanate component of the isocyanate prepolymer composition can include all essentially known aliphatic, cycloaliphatic, araliphatic and preferably aromatic multivalent isocyanates. Specific examples include: alkylene diisocyanates with 4 to 12 carbons in the alkylene radical such as 1,12-dodecane diisocyanate, 2-ethyl-1,4-tetramethylene diisocyanate, 2-methyl-1,5-pentamethylene diisocyanate, 1,4-tetramethylene diisocyanate and preferably 1,6-hexamethylene diisocyanate; cycloaliphatic diisocyanates such as 1,3- and 1,4-cyclohexane diisocyanate as well as any mixtures of these isomers, 1-isocyanato-3,3,5-trimethyl-5-isocyanatomethylcyclohexane (isophorone diisocyanate), 2,4- and 2,6-hexahydrotoluene diisocyanate as well as the corresponding isomeric mixtures 4,4′-2,2′- and 2,4′-dicyclohexylmethane diisocyanate as well as the corresponding isomeric mixtures and preferably aromatic diisocyanates and polyisocyanates such as 2,4- and 2,6-toluene diisocyanate and the corresponding isomeric mixtures 4,4′-, 2,4′- and 2,2′-diphenylmethane diisocyanate and the corresponding isomeric mixtures, mixtures of 4,4′- and 2,4′-diphenylmethane diisocyanates and polyphenylenepolymethylene polyisocyanates (polymeric MDI), as well as mixtures of polymeric MDI and toluene diisocyanates. The organic di- and polyisocyanates can be used individually or in the form of mixtures.

Frequently, so-called modified multivalent isocyanates, i.e., products obtained by the partial chemical reaction of organic diisocyanates and/or polyisocyanates are used. Examples include diisocyanates and/or polyisocyanates containing ester groups, urea groups, biuret groups, allophanate groups, carbodiimide groups, isocyanurate groups, and/or urethane groups. Specific examples include organic, preferably aromatic, polyisocyanates containing urethane groups and having an NCO content of 33.6 to 15 weight percent, preferably 32 to 21 weight percent, based on the total weight, e.g., with low molecular weight diols, triols, dialkylene glycols, trialkylene glycols, or polyoxyalkylene glycols with a molecular weight of up to 1500; modified 4,4′-diphenylmethane diisocyanate or 2,4- and 2,6-toluene diisocyanate, where examples of di- and polyoxyalkylene glycols that may be used individually or as mixtures include diethylene glycol, dipropylene glycol, polyoxyethylene glycol, polyoxypropylene glycol, polyoxyethylene glycol, polyoxypropylene glycol, and polyoxypropylene polyoxyethylene glycols or -triols.

Furthermore, liquid polyisocyanates containing carbodiimide groups having an NCO content of 33.6 to 15 weight percent, preferably 32 to 21 weight percent, based on the total weight, have also proven suitable, e.g., based on 4,4′- and 2,4′- and/or 2,2′-diphenylmethane diisocyanate and/or 2,4′- and/or 2,6-toluene diisocyanate. The modified polyisocyanates may optionally be mixed together or mixed with unmodified organic polyisocyanates such as 2,4′- and 4,4′-diphenylmethane diisocyanate, polymeric MDI, 2,4′- and/or 2,6-toluene diisocyanate.

Organic polyisocyanates which may be employed include aromatic, aliphatic, and cycloaliphatic polyisocyanates and combinations thereof. Representative of these types are the diisocyanates such as m-phenylene diisocyanate, 2,4-toluene diisocyanate, 2,6-toluene diisocyanate, mixtures of 2,4- and 2,6-toluene diisocyanate, hexamethylene diisocyanate, tetramethylene diisocyanate, cyclohexane-1,4-diisocyanate, hexahydrotoluene diisocyanate (and isomers), naphthalene-1,5-diisocyanate, 1-methoxyphenyl-2,4-diisocyanate, 4,4′-diphenylmethane diisocyanate, mixtures of 4,4′- and 2,4′-diphenylmethane diisocyanate, 4,4′-biphenylene diisocyanate, 3,3′-dimethoxy-4,4′-biphenyl diisocyanate, 3,3′-dimethyl-4,4′-biphenyl diisocyanate and 3,3′-dimethyldiphenylmethant-4,4′-diisocyanate; the triisocyanates such as 4,4′,4″-triphenylmethane triisocyanate, and toluene 2,4,6-trisocyanate; and the tetraisocyanates such as 4,4′-dimethldiphenylmethane-2,2′,5,5′-tetraisocyanate and polymeric polyisocyanates such as polymethylene polyphenylene polyisocyanate, and mixtures thereof.

Crude polyisocyanates may also be used in the compositions of the present invention, such as crude toluene diisocyanate obtained by the phosgenation of a mixture of toluenediamines or crude diphenylmethane isocyanate obtained by the phosgenation of crude isocyanates are disclosed in U.S. Pat. No. 3,215,652.

In a preferred embodiment, the polyisocyanate compound reacted with the polyhydroxyl compound of the present invention is a polymeric diphenylmethane diisocyanate (MDI) having a nominal functionality of from 2 to 3 , and an NCO content of from 30 to 35 weight percent.

A particularly preferred polyisocyanate compound of the present invention includes Lupranate® M20S, a polymeric MDI commercially available from BASF Corporation.

The isocyanate compound is mixed in stochiometric excess with the hydroxyl containing compound such that the formed isocyanate prepolymer composition has a free NCO content of at least 11% by weight.

Also included in the isocyanate prepolymer composition of the present invention is a plasticizer. Plasticizers are generally used to promote good processing characteristics of the base resin or improve the characteristics of the finished product, such as the flexibility of the product at a lower temperature. A preferred type of plasticizer that may be utilized by the present invention includes the phthalate class of plasticizers although other types of plasticizers may be used. The phthalate class of plasticizers is known in the art, and includes orthophthalate and terephthalate classes. A preferred plasticizer of the present invention is a linear phthalate plasticizer based upon C₇, C₉ and C₁₁ alcohols, under the name Palatinol® 711P and is commercially produced by the BASF Corporation. The plasticizer generally is present in an amount of from 2 to 15 weight % based on the overall weight of the isocyanate prepolymer composition.

Another component preferably included in the isocyanate prepolymer composition is an oil. As with the plasticizer outlined above, the oil is included in the composition to improve the processing characteristics of the composition, such as the viscosity and/or to improve the characteristics of the finished product. Generally the oil is present in an amount of from 2 to 15 weight % based on the total weight of the prepolymer composition.

Oils may be generally classified as refined or manmade products or natural oils that are produced by animals or plants. Examples of natural oils include soybean, corn sunflower and canola oils. Natural oils are prone to become rancid over time and are therefore less desirable than refined oils.

The refined oils include petroleum byproducts such as aliphatic, naphthenic and aromatic hydrocarbons as well as chlorinated hydrocarbons. A particularly preferred oil of the present invention is a naphthenic oil. The naphthenic class of oils are primarily three and four membered ring structures with pendant alkyl groups. A subset of the naphthenic class of oils includes the white mineral oil class of oils. The white mineral oils are viscous hydrocarbon liquids derived from petroleum. The oils are refined to remove constituents that impart odor, color taste and potential toxicological properties. The constituents removed include compounds containing sulfur, oxygen and nitrogen, as well as aromatics and olefins. The resultant oil contains almost entirely saturated hydrocarbons.

Another particularly preferred oil of the present invention is Hyprenee® L-100, a naphthenic white mineral oil produced by Ergon Refining. The oil is preferably present in an amount of from 3 to 10 weight % based on the overall weight of the isocyanate prepolymer composition.

Another component included in the isocyanate prepolymer composition of the present invention is a phosphate ester. Phosphate esters are produced as the reaction product of hydroxyl containing compounds with phosphoric acid. The phosphate ester hinders the isocyanates reaction with water in the ambient air and thereby extends the “potlife” of the prepolymer composition. The phosphate esters that are suitable for the present invention have the following characteristics: a pentavalent phosphorous atom, one phosphorous-oxygen double bond, 1 or 2 alkoxy or aryloxy groups, and one hydroxy group substituent. They have the following general formula:

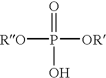

Wherein R′ and R″ represent alkyl, aryl, aliphatic or cycloaliphatic groups. One of the groups R′ or R″ can be a hydrogen. Some typical examples include phosphoric acid monobutyl ester; phosphoric acid dibutyl ester; phosphoric acid monophenyl ester; phosphoric acid diphenyl ester; phosphoric acid 2-butoxy- 1-ethyl ester; 2-ethylhexyl acid phosphate, cetyl acid phosphate, and stearyl acid phosphate.

Particularly preferred phosphate esters of the present invention include butyl acid phosphate, and Maphos 60 A, an aliphatic phosphate ester, both of which are commercially produced by the BASF Corporation. The phosphate ester generally is present in an amount of from 0.1 to 1.2 weight % based on the overall weight of the isocyanate prepolymer composition.

Another preferred component of the isocyanate prepolymer composition of the present invention is benzoyl chloride. As outlined earlier, benzoyl chloride is added to inhibit the reaction of the isocyanate with water in the ambient air. Due to its negative properties, the amount of benzoyl chloride is limited to a minor amount. The benzoyl chloride generally is present in amount of from 0 to 0.02 weight percent based on the overall weight of the isocyanate prepolymer composition.

The isocyanate prepolymer composition also preferably contains a flame retardant. Examples of suitable phosphate flameretarding agents are tricresyl phosphate, tris(2-chloroethyl) phosphate, tris(2-chloropropyl) phosphate, and tris(2,3-dibromopropyl) phosphate. In addition to these halogen-substituted phosphates, it is also possible to use inorganic or organic flameretarding agents, such as red phosphorous, aluminum oxide hydrate, antimony trioxide, arsenic oxide, ammonium polyphosphate (Exolit®) and calcium sulfate, molybdenum trioxide, ammonium molybdate, ammonium phosphate, pentabromodiphenyloxide, 2,3-dibromopropanol, hexabromocyclododecane, dibromoethyldibromocyclohexane, expandable graphite or cyanuric acid derivatives, e.g., melamine, or mixtures of two or more flameretarding agents, e.g., ammonium polyphosphates and melamine, and, if desired, corn starch, or ammonium polyphosphate, melamine, and expandable graphite and/or, if desired, aromatic polyesters, in order to flameretard the polyisocyanate polyaddition products. In general, from 2 to 40 percent, preferably from 5 to 20 percent, of said flameretarding agents may be used based on the weight of the isocyanate prepolymer composition.

As will be understood by one of ordinary skill in the art the present invention also finds utilization in extending the potlife of blends of isocyanate prepolymers.

The phosphate esters of the present invention are also useful for extending the potlife of an isocyanate or isocyanate blends, even if there are no isocyanate prepolymers present. In this embodiment the composition preferably includes: at least one isocyanate, a phosphate acid ester, and a plasticizer. Preferably, the composition includes additionally benzoyl chloride, an oil and flame retardants. All of the suitable components have been described above. The isocyanate blends can include any combination of the isocyanates described above with respect to the isocyanate prepolymers.

One use of the isocyanate prepolymer composition of the present invention is as an adhesive. As an adhesive, the isocyanate prepolymer composition is reacted with a second composition that includes water, a catalyst, and a surfactant. Preferably, the second composition comprises 95% by weight water, 4.5% by weight catalyst, and .5% by weight surfactant.

The catalysts used are, in particular, components that accelerate the reaction of the hydroxyl groups with the isocyanate groups. Suitable catalysts are organic metal compounds, preferably organic tin compounds such as tin (II) salts of organic carboxylic acids, e.g., tin (II) acetate, tin (II) octoate, tin (II) ethylhexanate and tin (II) laurate, and the dialkyltin (IV) salts of organic carboxylic acids, e.g., dibutyltin diacetate, dibutyltin dilaurate, dibutyltin maleate, and dioctyltin diacetate.

A particularly preferred catalyst useful in association with the isocyanate prepolymer of the present invention is dibutyltin dilaurate and is commercially produced under the trade name Dabco®-T by the Air Products Corporation.

Also included with the second composition when utilizing the isocyanate prepolymer of the present invention is a surfactant. Examples of suitable surfactants are compounds which serve to support homogenization of the starting materials and may also regulate the cell structure of the plastics. Specific examples are salts of sulfonic acids, e.g., alkali metal salts or ammonium salts of fatty acids such as oleic or stearic acid, of dodecylbenzene- or dinaphthylmethanedisulfonic acid, and ricinoleic acid; foam stabilizers, such as siloxaneoxyalkylene copolymers and other organopolysiloxanes, oxyethylated alkyl-phenols, oxyethylated fatty alcohols, pariffin oils, castor oil esters, ricinoleic acid esters, Turkey red oil and groundnut oil, and cell regulators, such as paraffins, fatty alcohols, and dimethylpolysiloxanes. The surfactants are usually used in amounts of 0.01 to 5% based on the weight of the second stream. A particularly preferred surfactant is Surfynol® TG commercially available from the Air Products Corporation.

The following examples are intended to illustrate, but in no way limit, the scope of the present invention. The components employed in the working examples are defined as follows:

PRE1 is a first prepolymer composition.

PRE2 is a second prepolymer composition.

ISO is a polymeric MDI.

Polyol A is Pluracol® 380, a TMP (trimethylolpropane) initiated propylene and ethylene oxide polyether polyol commercially available from BASF Corporation.

Control 1 is a prepolymer of ISO and a polyol with a free NCO content of approximately 11.0%, containing an excessive amount of >0.1 weight % of benzoyl chloride. This prepolymer has an acceptable “potlife”.

Control 2 is a prepolymer of ISO and a polyol with a free NCO content of approximately 11.0%, and no acid phosphate esters or benzoyl chloride. This prepolymer has an unacceptable “potlife”.

TCPP is a flame retardant, available from Great Lakes Chemical.

BAP is butyl acid phosphate, a phosphate ester of the present invention.

MAP is Maphos 60 A, an aliphatic phosphate ester of the present invention commercially produced by the BASF Corporation.

Plasticizer is Palatinol® 711P a phthalate based plasticizer commercially available from BASF Corporation.

Oil is Hyprene® L-100, a naphthenic white mineral oil produced by Ergon Refining.

CAT is a dibutyltin dilaurate catalyst and is commercially produced under the trade name Dabco®-T by the Air Products Corporation.

BEN is benzoyl chloride.

SURF is Surfynol® TG, a surfactant commercially available from the Air Products Corporation.

### EXAMPLE 1

The first and second prepolymer composition, PRE1 and PRE2 are formed by charging the ISO component into a five liter flask under agitation and heating it to 60° C. Polyol A is then added and the temperature is maintained at 80° C. for 2 hours. The reaction mass is then cooled to 70° C. at which time the BAP, Plasticizer, and Oil components are added under agitation. The reaction mass is then cooled to 50° C. and the TCPP is added under agitation. The resultant prepolymers are collected for further experimentation, the description of which is outlined below with the results shown in Tables 2 and 3. The formulas outlined in Table 1 show the amounts of the various components expressed as a weight percent based on the overall weight of the prepolymer.

TABLE 1

| Ingredients | PRE 1 | PRE 2 |
|---|---|---|
| Polyol A | 37.2 | 37.866 |
| ISO | 37.5 | 37.462 |
| BAP | 0.2 | 0.39 |
| TCCP | 12.5 | 12.589 |
| Plasticizer | 12.5 | 6.043 |
| Oil | 0.0 | 5.65 |
| BEN | 0.1 | 0.0 |

Prepolymer 1 (PRE 1) obtained per Table 1 and the two control prepolymers were then tested to determine the stability or reactivity in the presence of water. Varying amounts of phosphate esters were added to PRE 1 and the relative reactivity was measured as follows. Fifty grams of a sample of the prepolymer are added to a small paper cup. Small amounts of the phosphate esters are added to the prepolymer and the mixture is stirred for 10 to 20 seconds. Five grams of a catalyst solution, as defined in Table 2 are added to the cup, and the resulting mixture is stirred vigorously for 45 seconds. Time measurements in minutes and seconds, as shown in Table 3, are taken when the resulting foam reaches a height of 2 inches in the cup and when the resulting foam reaches a height of the rim of the cup. The longer the time required to rise the more stable the prepolymer composition is in the presence of water.

Based on the stability/reactivity test results for PRE 1, prepolymer 2 (PRE 2) is prepared as per Table 1, as a fully formulated prepolymer for potential commercial applications. The stability of PRE 2 is judged as acceptable by comparison with Control 1.

TABLE 2

| COMPONENT | AMOUNT WEIGHT % |
|---|---|
| Water | 95 |
| CAT | 4.5 |
| SURF | .5 |

TABLE 3

| | Total amount of MAP/BAP in | | |
|---|---|---|---|
| Prepolymer | the prepolymer in weight % | T (2 inches) | T (rim) |
| Control 1 | 0.0 MAP/0.0 BAP | 2 m 15 s | 2 m 46 s |
| Control 2 | 0.0 MAP/0.0 BAP | 1 m 25 s | 1 m 48 s |
| PRE 1 | 0.3 MAP/0.2 BAP | 2 m 10 s | 2 m 45 s |
| PRE 1 | 0.5 MAP/0.2 BAP | 3 m 10 s | 4 m 11 s |
| PRE 1 | 1.0 MAP/0.2 BAP (foam | 3 m 25 s | 4 m 38 s |
| | collapsed) | | |
| PRE 1 | 0.0 MAP/0.4 BAP | 2 m 06 s | 2 m 41 s |
| PRE 1 | 0.0 MAP/0.5 BAP | 3 m 14 s | 4 m 11 s |
| PRE 1 | 0.0 MAP/1.2 BAP | 3 m 30 s | 4 m 39 s |
| PRE 2 | 0.0 MAP/0.4 BAP | 2 m 14 s | 3 m 04 s |

As can be seen from the preceding table increasing the levels of phosphate ester in the prepolymer stabilizes the mixture toward reaction with water. Phosphate ester levels totaling from 0.4 to 1.2 weight % achieve acceptable stabilization as compared with the Control samples. It can also been seen from the above results that the BAP is more efficient on a weight basis as compared to the MAP at stabilizing the prepolymer in the presence of water. Also BAP has the advantage that high amounts can be used without the detrimental effect of causing the foam to collapse, as occurred with the high (1.0 weight %) level of MAP.

What is claimed is:

1. An isocyanate prepolymer composition comprising:
a. an isocyanate-terminated prepolymer;
b. a plasticizer; and
c. a phosphate ester
said phosphate ester having at least one acidic hydrogen and a pentavalent phosphorous atom.

2. The isocyanate prepolymer composition of claim 1, wherein said prepolymer comprises polymeric MDI.

3. The isocyanate prepolymer composition of claim 1, wherein the prepolymer comprises a polyetherol.

4. The isocyanate prepolymer composition of claim 3, wherein the polyetherol comprises a trimethylpropane initiated polyol comprising propylene oxide and ethylene oxide.

5. The isocyanate prepolymer composition of claim 1, wherein the prepolymer comprises a polyesterol.

6. The isocyanate prepolymer composition of claim 1, wherein said prepolymer composition has a free NCO content of at least 11 percent.

7. The isocyanate prepolymer composition of claim 1, wherein said plasticizer is present in an amount of from 2 to 15 weight percent based on the total weight of the prepolymer composition.

8. The isocyanate prepolymer composition of claim 1, wherein said plasticizer comprises a phthalate based plasticizer.

9. The isocyanate prepolymer composition of claim 1, further comprising an oil.

10. The isocyanate prepolymer composition of claim 9, wherein said oil comprises a naphthenic petroleum based oil.

11. The isocyanate prepolymer composition of claim 9, wherein said oil comprises a natural oil.

12. The isocyanate prepolymer composition of claim 9, wherein said oil is present in an amount of from 2 to 15 weight percent based on the total weight of the prepolymer composition.

13. The isocyanate prepolymer composition of claim 1, further comprising benzoyl chloride.

14. The isocyanate prepolymer composition of claim 13, wherein said benzoyl chloride is present in an amount of from 0.001 to 0.02 weight percent based on the total weight of the prepolymer composition.

15. The isocyanate prepolymer composition of claim 1, wherein said phosphate ester is present in an amount of from 0.1 to 1.2 weight percent based on the total weight of the prepolymer composition.

16. The isocyanate prepolymer composition of claim 1, wherein said phosphate ester comprises: phosphoric acid monobutyl ester; phosphoric acid dibutyl ester; phosphoric acid monophenyl ester; phosphoric acid diphenyl ester; phosphoric acid 2-butoxy-1-ethyl ester; 2-ethylhexyl acid phosphate; cetyl acid phosphate; stearyl acid phosphate; or mixtures thereof.

17. The isocyanate prepolymer composition of claim 1, wherein said phosphate ester comprises butyl acid phosphate.

18. The isocyanate prepolymer composition of claim 1, wherein said phosphate ester comprises Maphos 60 A.

19. The isocyanate prepolymer composition of claim 1, further including a flame retardant.

20. The isocyanate prepolymer composition of claim 18, wherein said flame retardant is present in an amount of from 5 to 20 weight percent based on the total weight of the prepolymer composition.

21. A polyisocyanate composition comprising:
a. a polyisocyanate;
b. a plasticizer; and
c. a phosphate ester
said phosphate ester having at least one acidic hydrogen and a pentavalent phosphorous atom.

22. The polyisocyanate composition of claim 21, wherein said polyisocyanate comprises polymeric MDI.

23. The polyisocyanate composition of claim 21, wherein said plasticizer is present in an amount of from 2 to 15 weight percent based on the total weight of the polyisocyanate composition.

24. The polyisocyanate composition of claim 21, wherein said plasticizer comprises a phthalate based plasticizer.

25. The polyisocyanate composition of claim 21, further comprising an oil.

26. The polyisocyanate composition of claim 25, wherein said oil comprises a naphthenic petroleum based oil.

27. The polyisocyanate composition of claim 25, wherein said oil comprises a natural oil .

28. The polyisocyanate composition of claim 25, wherein said oil is present in an amount of from 2 to 15 weight percent based on the total weight of the polyisocyanate composition.

29. The polyisocyanate composition of claim 21, further comprising benzoyl chloride.

30. The polyisocyanate composition of claim 29, wherein said benzoyl chloride is present in an amount of from 0.001 to 0.02 weight percent based on the total weight of the polyisocyanate composition.

31. The polyisocyanate composition of claim 21, wherein said phosphate ester is present in an amount of from 0.1 to 1.2 weight percent based on the total weight of the polyisocyanate composition.

32. The polyisocyanate composition of claim 21, wherein said phosphate ester comprises: phosphoric acid monobutyl ester; phosphoric acid dibutyl ester; phosphoric acid monophenyl ester; phosphoric acid diphenyl ester; phosphoric acid 2-butoxy-1-ethyl ester; 2-ethylhexyl acid phosphate; cetyl acid phosphate; stearyl acid phosphate; or mixtures thereof.

33. The polyisocyanate composition of claim 21, wherein said phosphate ester comprises butyl acid phosphate.

34. The polyisocyanate composition of claim 21, wherein said phosphate ester comprises Maphos 60 A.

35. The polyisocyanate composition of claim 21, further including a flame retardant.

36. The polyisocyanate composition of claim 35, wherein said flame retardant is present in an amount of from 5 to 20 weight percent based on the total weight of the polyisocyanate composition.
